(19) United States
(12) Patent Application Publication
(10) Pub. No.: US 2014/0107718 A1
(43) Pub. Date: Apr. 17, 2014

(54) AUTOMATED EXTERNAL DEFIBRILLATOR ATTACHMENT FOR ELECTRONIC DEVICE

(71) Applicant: HEARTLIFE TECHNOLOGY, LLC, Auburn, AL (US)

(72) Inventors: Thomas W. FOOTE, Auburn, AL (US); Thaddeus A. ROPPEL, Auburn, AL (US)

(73) Assignee: HEARTLIFE TECHNOLOGY, LLC, Auburn, AL (US)

(21) Appl. No.: 14/051,777

(22) Filed: Oct. 11, 2013

Related U.S. Application Data
(60) Provisional application No. 61/712,792, filed on Oct. 11, 2012.
(60) Provisional application No. 61/779,804, filed on Mar. 13, 2013.
(60) Provisional application No. 61/876,349, filed on Sep. 11, 2013.

(51) Int. Cl. A61N 1/39; A61N 1/04

(52) U.S. Cl. CPC A61N 1/3993; A61N 1/046
USPC 607/7; 607/142

(57) ABSTRACT

An automated defibrillator attachment for an electronic device such as a smart phone includes an electronics module, at least two electrodes, and a connector. The at least two electrodes extend from the electronics module for connecting to a patient. The connector extends from the electronics module for coupling to at least one receptacle of the electronics device. The at least two electrodes provide at least one electrical shock to the patient that is supplied from an energy source of the electronic device. Other embodiments include an energy storage element to supply the at least one electrical shock.

### CROSS-REFERENCE TO RELATED APPLICATIONS

This application claims priority benefit to U.S. Provisional Patent Application Ser. No. 61/712,792, filed Oct. 11, 2012, U.S. Provisional Patent Application Ser. No. 61/779,804, filed Mar. 13, 2013, and U.S. Provisional Patent Application Ser. No. 61/876,349, filed Sep. 11, 2013, all of which are hereby incorporated herein by reference in their entireties.

### TECHNICAL FIELD

The present invention relates generally to the field of medical devices and equipment, and more particularly to an automated external defibrillator attachment for portable electronic devices, and software for utilizing such an attachment.

### BACKGROUND

Myocardial infarctions and other heart disorders that lead to sudden cardiac arrest are becoming more prevalent in today's society. Generally, the most common cause of sudden cardiac arrest is a heart attack that results from either ventricular fibrillation (VF) or pulseless ventricular tachycardia (PVT). A patient undergoing VF or PVT generally has a severely abnormal or irregular heart rhythm that causes quivering of the heart's lower chambers and causes the heart to suddenly stop. When sudden cardiac arrest occurs, death or permanent damage may be avoided by treating the sudden cardiac arrest victim with high quality cardiopulmonary resuscitation (CPR) and early electrical defibrillation. While CPR is capable of treating a sudden cardiac arrest victim, early electrical defibrillation is preferred.

Automated external defibrillators (AEDs) are commonly used to provide electrical defibrillation, which can be operated by both competent bystanders and trained professionals. Typically, AEDs are known to be too large, bulky and expensive to be carried by many health conscious citizens or by smaller businesses, which results in their availability being very limited to the general public in many places. Some advances have been achieved to provide the general public with access to AEDs, for example, wherein AEDs are strategically placed in large public venues. Additionally, first response units (fire department, paramedics, etc.) are typically equipped with AEDs, but their efficacy often depends on the response time. Although this availability is an improvement over past decades, there is still a substantial lack of timely defibrillation evidenced by over 90% of cardiac arrest victims dying before reaching the hospital setting.

Thus it can be seen that needs exist for improved automated external defibrillators. It is to the provision of an automated defibrillator attachment for electronic devices meeting these and other needs that the present invention is primarily directed.

### SUMMARY

In example embodiments, the present invention provides an automated external defibrillator attachment for electronic devices. In one aspect, the present invention relates to an automated external defibrillator attachment for an electronic device. The electronic device includes an energy source and at least one receptacle or electronic coupling. The automated external defibrillator attachment includes an electronics module, at least two electrodes extending from the electronics module for application to a patient, and a connector extending from the electronics module for coupling to the at least one receptacle of the electronics device. The at least two electrodes connect to a human or animal patient in cardiac distress to deliver at least one electrical shock capable of cardiac defibrillation effect to the patient. In example embodiments, the energy delivered by the electrical shock is supplied from the energy source of the electronic device. In one form, the electronics module includes a current boosting element for receiving energy from the energy source of the electronic device and outputting the at least one electrical shock. In example embodiments, the current boosting element includes a supercapacitor, a battery, or a combination of both. In another form, the energy delivered by the at least two electrodes to administer the at least one electrical shock is supplied from an external energy source. In example forms, the electronics module includes an energy storage element for outputting the at least one electrical shock of an amplitude to treat a person in cardiac distress. In example forms, the energy level of the at least one electrical shock is between about 120-200 joules. Optionally, the electrical shock is supplied from the electronics module.

In another aspect, the invention relates to a portable external defibrillator attachment for an electronic device. The electronic device includes at least one receptacle and signal analysis software. The portable external defibrillator attachment includes an electronics module, at least two electrodes extending from the electronics module for connecting to a patient, a connector extending from the electronics module for being received by the at least one receptacle to communicate with the electronic device, and a recharging connector. The electronics module includes an energy storage element, a shock delivery module, a signal processing electronics module, control electronics module, and power management electronics module. The at least two electrodes can receive electrocardiographic signals from a patient. The electrocardiographic signals received by the at least two electrodes are input to the signal processing electronics of the electronics module to be amplified and filtered. The amplified and filtered signals are output from signal processing electronics and input to the electronic device to be processed by the signal analysis software to determine whether the patient has a shockable rhythm. The shock delivery module receives energy from the energy storage element to deliver at least one electrical shock through the at least two electrodes and to the patient. The energy level of the at least one electrical shock is preferably between 120-200 joules.

In yet another aspect, the invention relates to defibrillator attachment for connecting to an electronic device. The electronic device includes a receptacle and software for guiding a user, performing analysis of an input signal, and outputting an output signal. The attachment includes defibrillator pads, a signal conditioning subsystem, and a shock delivery subsystem. The defibrillator pads connect to the signal conditioning subsystem, the signal conditional subsystem connects to the electronic device, and the shock delivery subsystem connects to the electronic device and the defibrillator pads. The signal conditioning subsystem receives electrocardiographic signals from a patient that comprise the input signals to be analyzed by the software to determine if the patient is in ventricular fibrillation. The output signal is received by the shock delivery subsystem to trigger outputting at least one electrical shock to the defibrillator pads. The shock delivery subsystem receives energy from an energy storage element to deliver at least one electrical shock through the defibrillator pads and to the patient. The energy level of the at least one electrical shock is between 120-200 joules.

In another aspect, the invention relates to an automated external defibrillator including an electronic device, an electronics module, at least two electrodes and a connector. The electronic device includes at least one receptacle and a signal analysis software. The electronics module includes an energy storage element, a shock delivery module, signal processing electronics module, control electronics module, and power management electronics module. The at least two electrodes extend from the electronics module for connecting to a patient. The connector extends from the electronics module for being received by the at least one receptacle of the electronics device to communicate with the electronic device. In example forms, the electronic device is selected from a cell phone, smart phone, MP3 player or other music player and/or video player, electronic reader, tablet computer, handheld game device, or the like. In one form, the electronic device is a smart phone.

In yet another aspect, the invention relates to a method of treating a patient undergoing cardiac arrest. The method includes providing an electronic device; providing a defibrillator attachment, the defibrillator attachment including an electronics module and at least two electrodes; connecting the defibrillator attachment to the electronic device; applying the at least two electrodes to the patient; receiving EKG signals from the patient; and outputting at least one electrical shock from the defibrillator attachment to the at least two electrodes.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of an automated defibrillator attachment according to an example embodiment of the present invention, and showing an example portable electronic device for attachment therewith.

FIG. 2 is a perspective view of an automated defibrillator attachment according to another example embodiment of the present invention.

FIG. 3 is a top schematic view of the electronics module of the automated defibrillator attachment of FIG. 2, wherein portions are removed to show internal portions thereof.

FIG. 4 is a perspective view of the automated defibrillator attachment of FIG. 2, showing the electronics module connected to a power outlet for recharging.

FIG. 5 is a perspective view of an automated defibrillator attachment according to another example embodiment of the present invention, showing the defibrillator attachment connected to the electronic device.

FIG. 6 is a front perspective view of a defibrillator attachment incorporated in a case according to another example embodiment of the present invention, the case at least partially housing the electronic device.

FIGS. 7A-B are rear perspective views of the case of FIG. 6, and showing two coupling members for removably coupling to the rear side of the case and comprising the defibrillation pads therein.

FIG. 8A is a perspective view of a defibrillator attachment incorporated in a case according to yet another example embodiment of the present invention.

FIG. 8B is a perspective view of the front half of the case shown in FIG. 8A.

FIG. 8C is a perspective view of the back half of the case shown in FIG. 8A.

FIG. 9A is a perspective view of a defibrillator pad according to another example embodiment of the present invention, showing the pad in a packaged and wrapped configuration.

FIG. 9B is a plan view of the pad of FIG. 9A, showing the packaging and wrapping configuration and showing an example operation of unwrapping the same.

FIG. 10A is a perspective view of a defibrillator pad according to another example embodiment of the present invention, showing the pad in a packaged and wrapped configuration.

FIG. 10B is a perspective view of the pad of FIG. 10A, showing the packaging removed therefrom and showing the pad and cover arranged in a parachute-like manner.

FIG. 10C is a perspective view of the pad of FIG. 10A, showing the packaging and folding configuration and showing an example operation of removing the pad from the cover.

FIGS. 11A-F show a defibrillator attachment incorporated in a case according to an additional example embodiment of the present invention, showing components thereof that are capable of wirelessly connecting to the electronics device and electrically coupling to the defibrillator pads.

FIG. 12 shows a defibrillator attachment according to yet another example embodiment of the present invention, showing components thereof that are capable of wirelessly connecting therebetween and/or wirelessly connecting to the electronics device.

FIG. 13 is a flow diagram of an example method of treating a patient undergoing cardiac arrest according to another example embodiment of the present invention.

FIGS. 14A-B show schematic diagrams of the defibrillator attachment according to additional example embodiments of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

Generally described, the present invention provides an automated external defibrillator attachment comprising a system of components which can serve to provide access to automated cardiac defibrillation for the general public at lower cost and at more locations than is presently the case. Moreover, the present invention seeks to save lives by leveraging the widespread availability of portable electronic devices, and when these portable electronic devices are attached or synched with the present invention (wired connection or wireless connection), an automated external defibrillator is provided. U.S. Provisional Patent Application Ser. No. 61/712,792, filed Oct. 11, 2012, U.S. Provisional Patent Application Ser. No. 61/779,804, filed Mar. 13, 2013, and U.S. Provisional Patent Application Ser. No. 61/876,349, filed Sep. 11, 2013 are incorporated herein by reference, and show example embodiments of automated external defibrillator attachments.

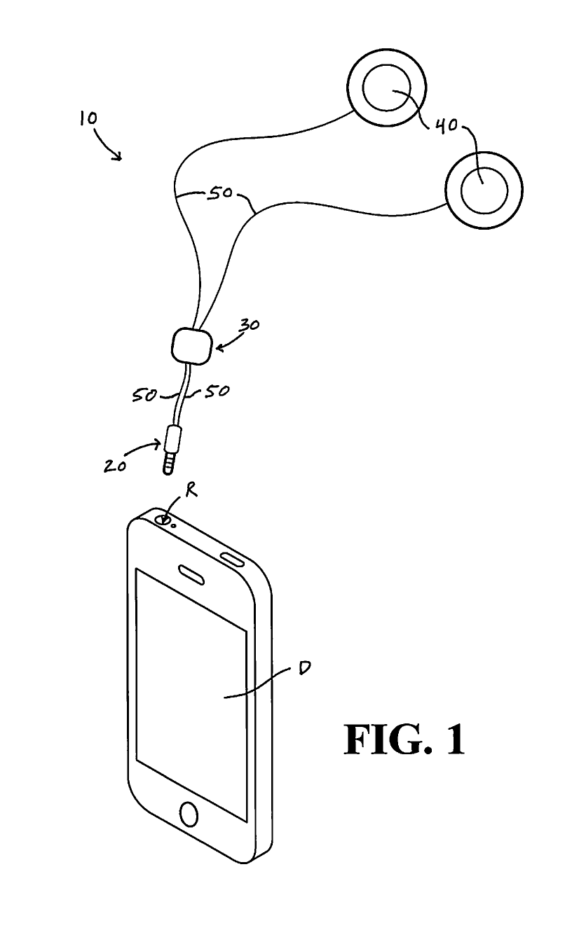

With reference now to the drawing figures, wherein like reference numbers represent corresponding parts throughout the several views, FIG. 1 shows an automated external defibrillator attachment 10 according to an example embodiment of the present invention. The defibrillator attachment 10 preferably connects to a portable electronic device D to treat a patient undergoing cardiac arrest, for example, to deliver an electrical shock to the patient through a pair of defibrillator pads or electrodes 40. The electronic device D may, for example, take the form of a cell phone, smart phone, MP3 player or other portable music and/or video player, electronic reader, tablet computer, handheld game device, or the like. Typically, the electronic device D comprises an energy storage element or battery that may be utilized with the present invention. Common types of batteries can include lithium ion batteries, traditional alkaline batteries, silver-oxide or silver zinc batteries, etc. As will be described below, some embodiments of the present invention allow for at least a portion of the electrical shock to be provided by the battery of the electronic device D. In other example embodiments, an energy storage element of an electronics module can provide for 100% of the electrical shock, thus using the electronic device as a user interface (UI) or to display data for analyzing and/or prompting the electronics module to deliver at least one electrical shock.

In example embodiments, the electronic device D comprises input means such as a keyboard or touch-screen, output means such as a display or audible output, a microprocessor and computer readable media or memory having signal analysis software resident therein, rhythm recognition software, supervisory software, and/or a program or application for communicating with the automated external defibrillator attachment 10, which software is executable by the processor. Generally, the supervisory software operates to receive input signals from the defibrillator attachment 10, analyze and process the input signals, and depending on the analysis of the input signals, output signals or electrical current from the electronic device D. Thus, with the defibrillator attachment 10 connected to the electronic device D, the supervisory software operates to communicate therewith wherein a patient undergoing cardiac arrest can be treated with defibrillation (e.g., electrical shocks).

As depicted in FIG. 1, the automated defibrillator attachment 10 generally comprises a connector 20, an electronics module 30, the pair of electrodes or defibrillator pads 40, and electrical conductors such as wiring 50 connected therebetween. Generally, the wiring 50 connects the connector 20 to the electronics module 30, and connects the electronic module 30 to the electrodes 40. Preferably, the wiring 50 can comprise a plurality of wires to provide for both inputting and outputting signals and/or electrical current to/from the electronic device D. For example, the wiring 50 provided for connecting the electrodes 40 to the electronics module 30 can comprise at least two wires for each of the electrodes 40 to provide for both receiving input signals therefrom and outputting electrical current to the electrodes 40 to provide for the electrical shock. Optionally, one wire is provided to connect each electrode 40 to the electronics module 30. In another embodiment, each wiring 50 connecting the connector 20 to the electronics module 30 (shown as two) comprises at least two wires to provide for both inputting the input signal through an audio port or receptacle R (microphone input) of the electronic device D, and to output the output signals (electrical current or signal) from the electronic device D through an audio output port or receptacle R (headphone output) to the electronics module 30.

The connector 20 preferably connects to the receptacle R of the electronic device D. In one form, the connector 20 is a 3 ring 3.5 millimeter jack and the receptacle R for receiving the connector 20 is the audio or headphone port (having a microphone input and headphone output). Optionally, other connectors having one or more wires (for use with cooperating receptacles) can be used as desired, for example, USB, mini USB, Dock connector 30-pin (Apple™), Lightning™ 8-pin (Apple™), Thunderbolt™, and other connectors. Preferably, the connector 20 can function to both send signals to the receptacle (to be input to the electronic device D and processed by the supervisory software) and receive signals from the receptacle (to be output from the electronic device D to the defibrillator attachment 10). Optionally, a new connector and cooperating receptacle may be developed to be used specifically for the automated external defibrillator attachment 10. Further, the connector 20 may be integrated within the electronics module 30 such that the defibrillator attachment 10 communicates with the electronic device D wirelessly (as will be described below). The wireless communication means may be in the form of USB, Bluetooth, WiFi, IR, or others as desired.

The electronics module 30 is generally provided to receive the output signals or electrical current that is output from the electronic device D such that the electrical shock can be generated and delivered to the electrodes 40. The electronics module 30 is generally external or separate from the electronic device D, and is connected thereto with the connector 20. As described above, the connection between the electronics module 30 and the electronic device D can be a wired connection or a wireless connection. In one form, the electronics module 30 is in the form of a current boosting element or supercapacitor. Preferably, the current boosting element can boost, multiply or magnify the energy that is output therefrom relative to the energy that is input (energy from the battery supply of the electronic device). Thus, the energy input into the current boosting element is relatively small compared to the energy output from the current boosting element. For example, for the current boosting element to produce a total energy level of X Joules, the supervisory software will trigger the electronic device D to output energy from the battery of a magnitude of about ½X Joules, more preferably about ⅛X Joules, more preferably about 1/16X Joules, and more preferably about 1/32X Joules. Typically, the total energy level produced by the current boosting element is generally between about 120-200 Joules. Generally, the energy input into the current boosting element (controlled by the supervisory software) is configured such that the current boosting element outputs or produces a voltage waveform according to the accepted standard biphasic pattern used in modern defibrillators. As such, the numerical values of the voltage and current output from the current boosting element will depend upon the total energy level (measured in Joules) that is determined by the supervisory software, and upon the resistance existing between the electrodes 40 connected to the patient. Moreover, if the electronics module 30 is connected to the electronic device D wirelessly, the electronics module 30 (as described below) is capable of comprising an energy storage element that can output electrical current therefrom to deliver an electrical shock. In example forms, to produce a total energy level of about 120-200 joules, the voltage range is between about 500-2000 volts, the current range is between about 10-30 amps, and the time span over which the electrical shock is delivered is between about 4-12 milliseconds. Optionally, other voltages, currents and time spans can be used as desired.

The defibrillator pads or electrodes 40 are generally biphasic such that biphasic algorithms can be utilized when shocking the patient undergoing cardiac arrest, for example, wherein each shock moves in an opposite polarity between the electrodes 40. Preferably, the electrodes 40 can function similarly to defibrillator pads of commercially available automated external defibrillator units, and typically comprise an adhesive or other coupling means for attaching to the patient's body. To allow the supervisory software to analyze the heart rhythm or electrocardigraphic (EKG) signals of a patient, the electrodes 40 are compatible to each comprise an input and an output. For example, the input can be used for receiving the EKG signals from the patient (where the pads are applied) and the output can be used for delivering the electrical shock(s).

The EKG signals received by the pads are input into the electronic device D wherein the supervisory software determines if the signals signify a shockable rhythm. Example forms of shockable rhythms can include ventricular fibrillation (VF) having an amplitude less than 0.2 mV, fine ventricular fibrillation (FVF) having an amplitude greater than 0.1 mV and less than 0.2 mV, and ventricular tachycardia (VT) of single morphology (monomorphic VT) or several morphologies (polymorphic VT). Generally, VT with a rate greater than 150 bpm and sustained for six seconds signifies a schockable rhythm. Preferably, the supervisory software comprises a plurality of stored algorithms consisting of the shockable rhythms to compare with the input signals. If the supervisory software determines that the EKG signals are a shockable rhythm, the supervisory software specifies the required energy level for the shock to be delivered to the electrodes 40 and prompts the electronic device D battery to output energy therefrom. The energy output from the electronic device D travels through the connector 20 and to the electrical module 30 to be multiplied or increased to produce a voltage waveform according to the accepted standard biphasic pattern used in modern defibrillators. This voltage waveform is generally between 120-200 Joules. Optionally, a monophasic pattern can be used to produce a voltage waveform between 200-360 joules. Preferably, the supervisory software is capable of calculating the level of energy that is output from the electronic device D battery such that the voltage waveform produced by the electronics module 30 is substantially similar to the required energy level specified by the supervisory software. Thus, the supervisory software can be adjusted accordingly depending on the particular specifications of the electronics module 30. Additionally, the supervisory software may identify detrimental rhythms that need correction in the form of smaller joule dosage. Rhythms such as extreme sinus tachycardia and bradycardia require 30-100 joules in a synchronized fashion known as pacing for optimal cardiac output. These rhythms are an indicator for an implantable pacemaker. If the supervisory software determines that the input signals does not signify a shockable condition, the supervisory software will cause the electronic device to display information notifying the user of the defibrillator attachment 10 that the patient does not have a rhythm that is shockable and the software does not provide a signal to prompt the electronic device D to output a signal or energy from the battery. Preferably, the supervisory software provides guidance to the user operating the defibrillator attachment 10 throughout the process, which may be in any combination of appropriate modes, such as visual, auditory, or tactile (e.g., vibrating to provide CPR cues, etc.).

Optionally, depending on the specifications of the electronics module 30, the supervisory software can prompt the electronic device D to rapidly discharge the battery wherein little to no multiplication or magnification of the energy received therefrom is necessary to provide the electrical shock. Preferably, batteries of all types that are used with current electronic devices can provide for supplying energy to the electronics module to provide for generating an electrical shock.

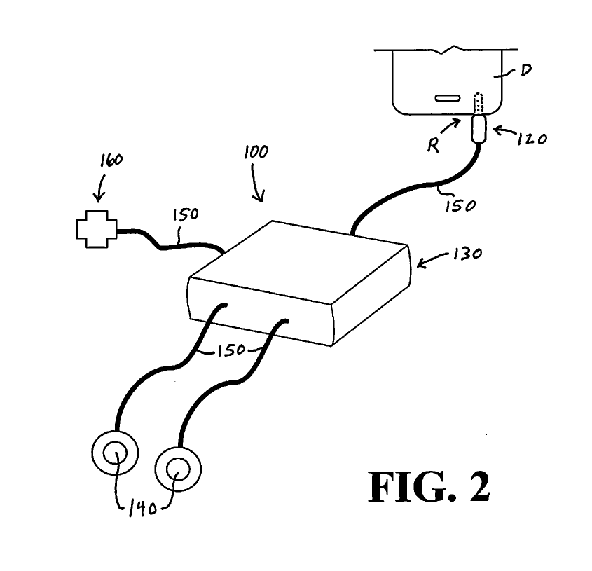

FIG. 2 shows an automatic defibrillator attachment 100 according to another example embodiment of the present invention. As depicted, the defibrillator attachment 100 comprises a connector 120 for connecting the attachment 100 to the electronic device D, an electronics module 130, a pair of electrodes 140, a universal recharging connector 160, and wiring 150 connected therebetween. Generally, the wiring 150 connects the connector 120 to the electronics module 130, connects the electrodes 140 to the electronics module 130, and connects the universal recharging connector 160 to the electronics module 130. Preferably, the wiring 150 can comprise a plurality of wires to provide for both inputting and outputting signals and/or electrical current to/from the electronics module 130. For example, the wiring 150 provided for connecting the electrodes 140 to the electronics module 130 comprises one wire for each of the electrodes 140 to provide for both receiving input signals (e.g., EKG signals) therefrom and outputting electrical current to the electrodes 140 to provide for the electrical shock. Thus, each wire extending from the electrode 140 to the electronics modules 130 provides the medium for both receiving the EKG signals and delivering the electrical shock. The wiring 150 connecting the connector 120 to the electronic module 130 comprises at least two wires to provide for both inputting the input signal through the audio input port or receptacle R (microphone input) of the electronic device D, and to output the output signals from the electronic device D through the audio output port or receptacle R (headphone output) to the electronics module 130. Optionally, as described above, other connectors having two or more wires can be used within corresponding receptacles of the electronic device D. The wiring connecting the universal recharging connector 160 is generally in the form of an integrated charging cable. Optionally, other wiring configurations can be used as desired.

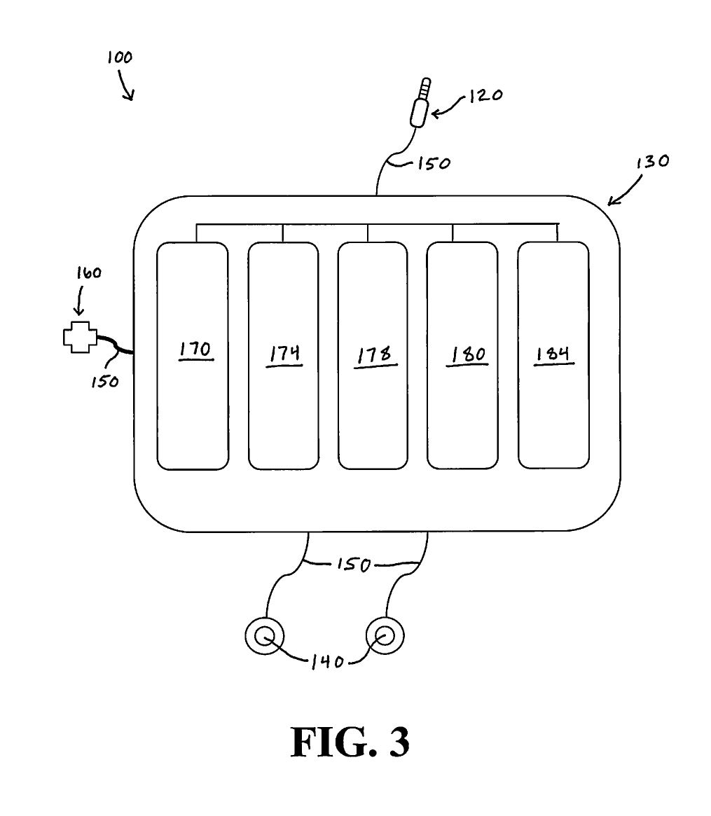

FIG. 3 shows an internal view of the electronics module 130 comprising a plurality of components to provide for automatic external defibrillation when the attachment 100 is connected to the electronic device D (running the supervisory software). As depicted, the electronics module 130 comprises an energy storage element 170, a shock delivery module 174, a signal processing electronics module 178, a control electronics module 180, and a power management module 184. The energy storage element 170 generally comprises a battery (lithium ion, lithium air, sodium air, alkaline, etc.), a supercapacitor, a combination of both a supercapacitor and a battery, or other form of current boosting element, or combinations thereof. The shock delivery module 174 generally comprises an electrical circuit that is configured to receive an output signal from the electronic device D (traveling through the connector 120) to operate in conjunction with the energy storage element 170 to generate an electrical shock that is sent to the electrodes 140. Generally, the electrical shock has an energy level and waveform that are determined by current standards and practices, for example, about 120-200 Joules. Preferably, the electrical shock is in all ways functionally identical to electrical shocks produced by current or known automated external defibrillators and in most every case approved by the Food and Drug Administration (FDA). The signal processing electronics module 178 provides for amplification and filtering of the raw EKG signals that are received or input into the electrodes 140. For example, after applying the electrodes 140 to the patient's chest, the EKG signals from the patient are input into the electrodes wherein they are amplified and filtered by the signal processing electronics module 178. The amplified and filtered signals from the signal processing electronics module 178 are then output through the connector 120 to be analyzed and processed by the supervisory software running on the electronic device D. Preferably, the output of the signal processing electronics module is compatible with the standard microphone-level inputs. As described above, other connectors can be used such that the signal processing module is compatible with the standard level inputs of other connectors. Additionally, the signal processing electronics module 178 is capable of synchronizing the at least one electrical shock with the rhythm (EKG signal) to ensure appropriate treatment and for avoiding further arrhythmia.

Generally, the amplification required is on the order of 40 to 60 dB, with common-mode rejection of typically 100 dB or greater. The filter is generally a bandpass type, with a passband of about 0.5 Hz to about 40 Hz. In one form, the signal processing electronics module 178 comprises a differential-mode input and a single-ended (ground-referenced) output. Further, additional filtering can be provided to reduce or eliminate electromyography (EMG) noise and/or electromagnetic interference (EMI). Generally, the EMG noise (electrical signals generated by movement of muscles other than the heart) and EMI may cause interference with the rhythm (EKG signal), thus it is desired that unwanted noise and/or interferences be filtered. The control electronics module 180 generally determines the operating mode of the attachment, for example, which can include a standby mode (low power requirement), a charging mode (to recharge the energy storage element 170), and an active mode (ready to operate). The power management electronics module 184 is generally provided to sense the charge level of the energy storage element 170 to determine if it is capable of delivering or providing the required shock energy, and to supervise the battery during charging. For example, it is known that rechargeable batteries (e.g., lithium ion batteries, etc.) require an intelligent charging system to avoid damage. Optionally, instead of a hard wiring or directly connecting the universal charging connector 160 to the electronics module 130 (via wiring 150), a separate universal charging connector comprising an inductive charging module may be included and the energy storage element 170 can be capable of induction charging, for example wherein simply setting the electronic module 130 atop or near the inductive charging module will permit charging of the energy storage element 170.

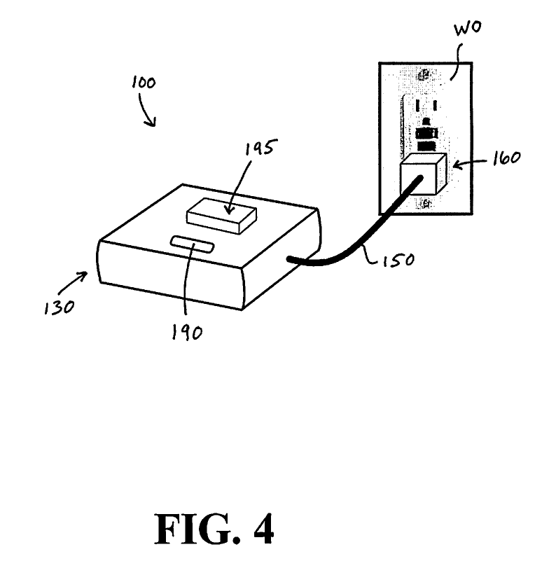

FIG. 4 shows the automated defibrillator attachment 100 connected to a power outlet or wall outlet WO to provide for charging of the energy storage element 170. As depicted, the universal charging connector 160 is connected to a standard AC power outlet WO. Preferably, a portion of the electronics module can comprise a charge level indicator 190 to provide the current level of the energy storage element 170. The charge level indicator 190 can be in the form of an array of lights, a single light (switching between an uncharged color and a charged color), or other forms of visual or audible feedback. The electronics module 195 can also comprise a storage compartment 195 for storing the electrodes 140, the connector 120, operating instructions and any other accessories that may be desirable to store therein. Optionally, the universal recharging connector 160 can comprise separable replacement connectors or accessories (12 V adapter plug or cigarette lighter plug for automobile, USB, etc.) for charging in other environments or locations. Preferably, the universal charging connector 160 can be connected to an assortment of power sources for recharging the energy storage element 170.

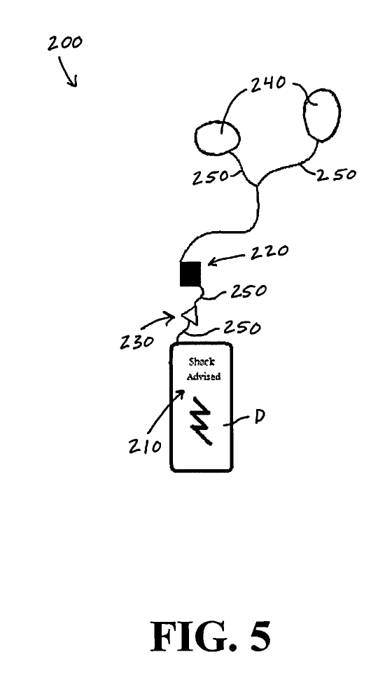

FIG. 5 shows an automated defibrillator attachment 200 according to another example embodiment of the present invention. Generally, the electronic device D comprises a lithium battery and is capable of downloading software applications from a marketplace, the internet or having them uploaded manually, for example, a rhythm recognition software or supervisory software 210. The device may also acquire the software during a developer update or it may be installed during initial manufacturing. In some forms, the battery may be used to provide a power source for the automated external defibrillator 200. As current medical practices recommend a shock dosage of 120, 150 and 200 joules for defibrillation, it is feasible to use a phone's battery, for example as some are capable of holding about 20,000 joules when fully charged. The supervisory software 210 is generally displayed and usable on the electronic device D. Defibrillation is generally recommended for the heart arrhythmias ventricular fibrillation and pulseless ventricular tachycardia. In some example forms, a safety feature is provided by not allowing shocks to be given to any patient who is not experiencing said arrhythmias.

In one form, an extra battery source 220 may be used in conjunction with the phone's battery to provide the at least one electrical shock, for example in case of a low battery or inability of the battery to discharge quickly enough. Relying on the external battery for shocking allows the electronic device to be used for rhythm diagnostic purposes through the supervisory software 210. In some example forms, the external battery is not required for the defibrillator attachment to operate. A capacitor or other resistance increasing device 230 may be provided to deliver joules at a rate higher than the rate at which batteries are generally accustomed to discharging. This may be needed to ensure therapeutic shocks are delivered in a timely fashion. The defibrillation pads 240 can be biphasic or monophasic. In some embodiments, the pads 240 may be changed from those currently on the market in order for the smaller device to receive appropriate electrical signals used to interpret ventricular fibrillation or ventricular tachycardia. Generally, the wiring 250 is provided for connecting the extra battery source 220, the capacitor 230, and the defibrillation pads 240.

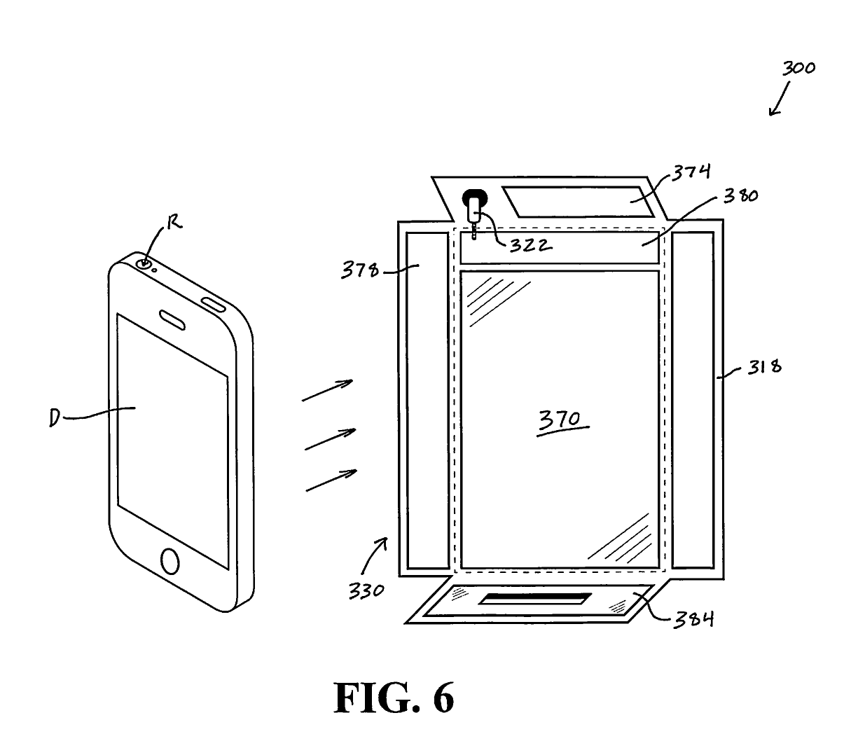

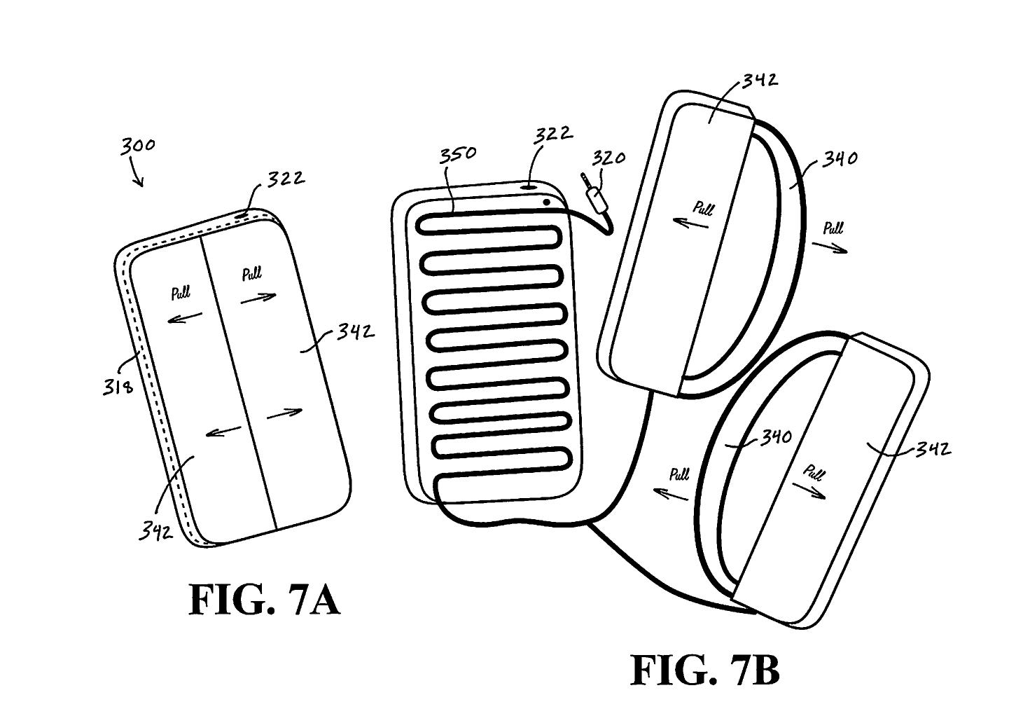

In additional example embodiments, the defibrillator attachment is combined with an electronic device case. Thus, with the electronic device D at least partially housed within the electronic device case, the defibrillator attachment is conveniently accessible and ready to use. In some forms, the electronic device case comprises an energy storage element that can be used to charge the electronic device D and/or for supplying the energy that is necessary to deliver the electrical shock. Preferably, all the components of the defibrillator attachment are housed inside or within the case such that they can be easily removed therefrom when treating a patient undergoing cardiac arrest. In one form, a recessed pocket is provided for securing the defibrillation pads therein. Optionally, other pockets, closures, and other features may be proved with the case to accommodate its use with the defibrillator attachment. For example, FIGS. 6-7B show a defibrillator attachment 300 combined with a case 314 according to another example embodiment of the present invention. The case 314 is generally capable of at least partially housing the electronic device D and comprises the components as described above to deliver the at least one electrical shock. Generally, the example embodiment shown in FIGS. 6-7B shows a wired configuration wherein the defibrillator pads comprise a wired connection, which is connected to the case or electronic device D to deliver the electrical shock. Referring to FIG. 6, the case 314 is generally formed from a shell or outer member 318 that defines an area wherein at least a portion of the electronic device D is housed. The top end of the case 414 generally includes an extension connector 322 that comprises a male end and a female end. In one example form, the extension connector 322 is at least partially housed within the shell 318 wherein the male end can be inserted into the receptacle R of the electronic device D when the device D is at least partially housed within the case 314. The female end is provided near the top end of the case 314 of the shell 318 for receiving an additional connector, for example to listen to music or other functions or operations that can be output from the electronic device D. Preferably, the shell 318 or other portions of the case at least partially house the electronics module 330 and the defibrillator pads 340. In example forms, the electronics module 330 generally includes the energy storage element 370, a shock delivery module 374, a signal processing electronics module 378, a control electronics module 380 and the power management module 384. Optionally, additional space can be provided for other electronics that can be used in addition to the electronics module 330. In one example form, an emergency battery to be used for powering the electronic device D and for providing defibrillation when the energy source of the electronic device has been depleted (the electronic device is dead, but by connecting the case to the electronic device the electronic device is still capable of powering signal analysis software and at least one shock). As depicted in FIGS. 7A-B, the back side of the case 314 comprises two generally elongate coupling members 342 for removably coupling to the back side of the case 314 and for housing the defibrillation pads 340 therein. Optionally, the outer contours of the coupling members 342 generally align with an outer contour of the shell 318 of the case 314. In one example form, wiring 350 extends between the defibrillator pads and a connector 320 for inserting into the female end 322 of the extension connector 322. Optionally, the wiring 350 can be configured to position itself such that in one configuration the wiring 350 is neatly secured to the case 314 and in another configuration the wiring 350 can extend therefrom (while remaining connected thereto) such that the pads can reach the patient undergoing cardiac arrest.

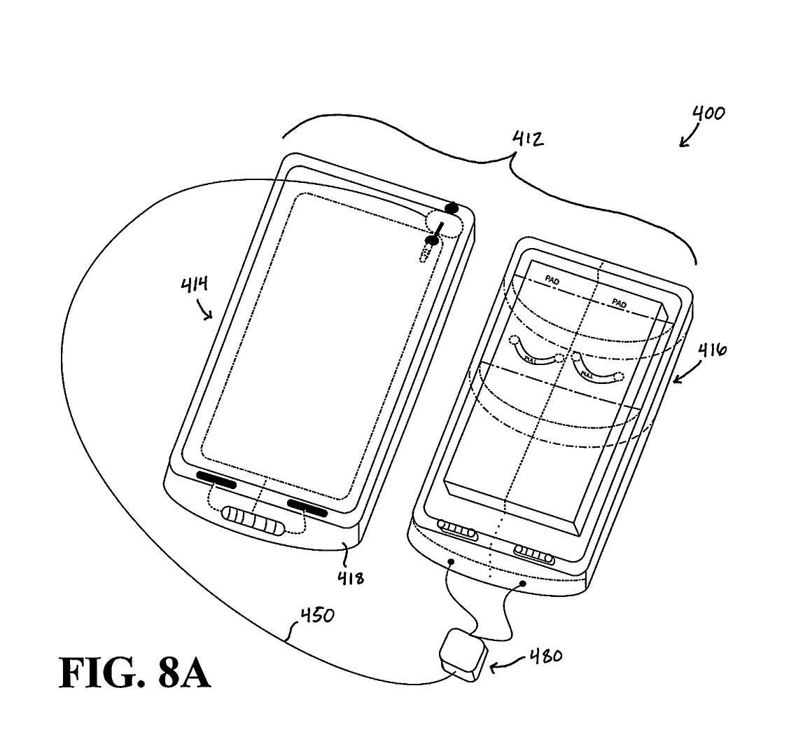

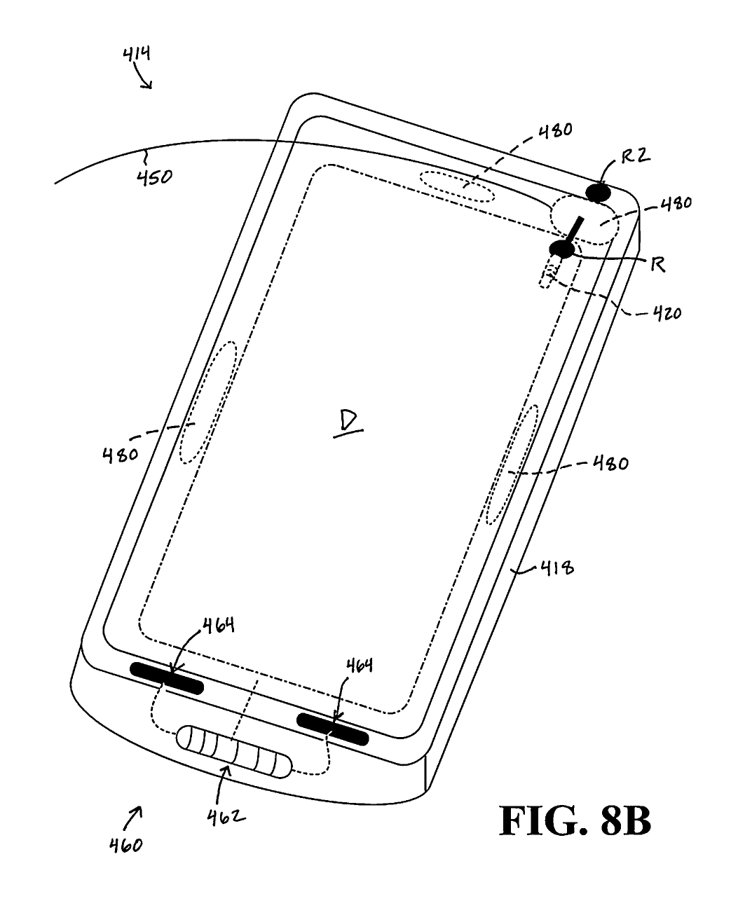

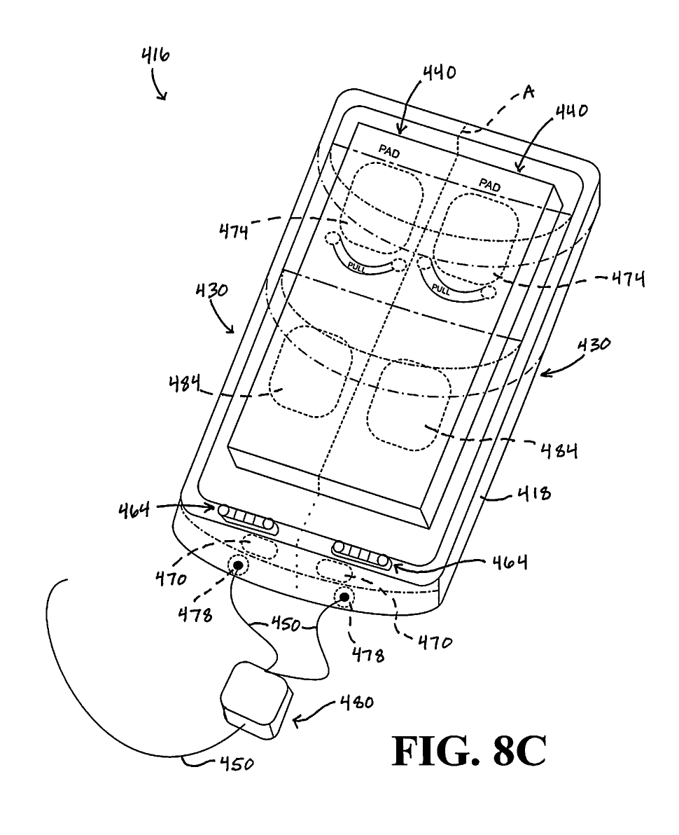

In yet another example embodiment, the present invention relates to a defibrillator attachment 400 combined with a releasable case 412. As depicted in FIGS. 8A-C, the releasable case 412 is at least partially housing the electronic device D and comprises the defibrillator attachment in order to provide at least one electrical shock to treat a patient undergoing cardiac arrest. In example forms, the releasable case is separable into at least two pieces along a generally horizontal plane that defines a front half 414 (FIG. 8A) and a back half 416 (FIG. 8B). In example forms, wiring 450 is attached to the front half 414 and extends to a control electronics module 480, which is connected to the back half 416 of the case 412. One skilled in the art would appreciate that the releasable case 412 is capable of being configured to separate along the length of the case to define a top portion and a bottom portion, or to separate in a variety of other configurations as desired. The front half 414 is generally formed from a shell or outer member 418 defining a chamber, reservoir or capturing or retaining member for at least partially receiving the electronic device D therein. The bottom end of the front half 414 generally includes a plurality of universal charging connectors 460. In one example form, a generally centrally-positioned universal charging connector 462 is formed in the shell 418 that comprises a male end and a female end. The male end is configured for connecting to a receptacle (unshown) of the electronic device when the electronic device D is at least partially housed within the case 412. The female end generally extends opposite of the male end and is positioned generally adjacent the shell 418 to provide for receiving the cooperating male end of a universal charging connector, which can be plugged into a power outlet. A pair of female universal charging connectors 464 are provided near the centrally-positioned charging connector 462 for cooperating with a pair of male universal charging connectors 464 of the back half 416 (see FIG. 8C). Preferably, when the front and back halves 314, 316 are releasably coupled together, the electrical power being input into the centrally-positioned universal charging connector 462 permits electrical power to flow into the electronic device (e.g., to charge the device's D battery) and to flow to the pair of female charging connectors 464 and through the cooperating pair of male charging connectors 464 of the back half 416 to charge energy storage elements 470 of each pad 440. The front half 414 of the case 412 may additionally include a connector 420 for inserting into a receptacle R of the electronic device D. Optionally, an additional receptacle R2 is provided near the top end of the front half 414 of the shell 418 for receiving an additional connector, for example to listen to music, or other functions that can be output from the electronic device D.

FIG. 8C shows greater detail of the back half 416 of the case 412. As depicted, the back half 416 is provided for separating into two pieces or components to form defibrillator pads 440. Generally, the back half comprises two electronics modules 430 including the energy storage element 470, the shock delivery module 474, the signal processing electronics module 478, the control electronics module 480, and the power management module 484. Preferably, these components are integrated and contained within the back half 316 of the case 412, for example wherein they are at least partially housed within the shell 418. The defibrillator pads 440 are mounted to a portion of the back half 416 such that they can be easily removed therefrom when desired. In example forms, the two pieces of the back half 416 are releasably coupled together along a central axis A.

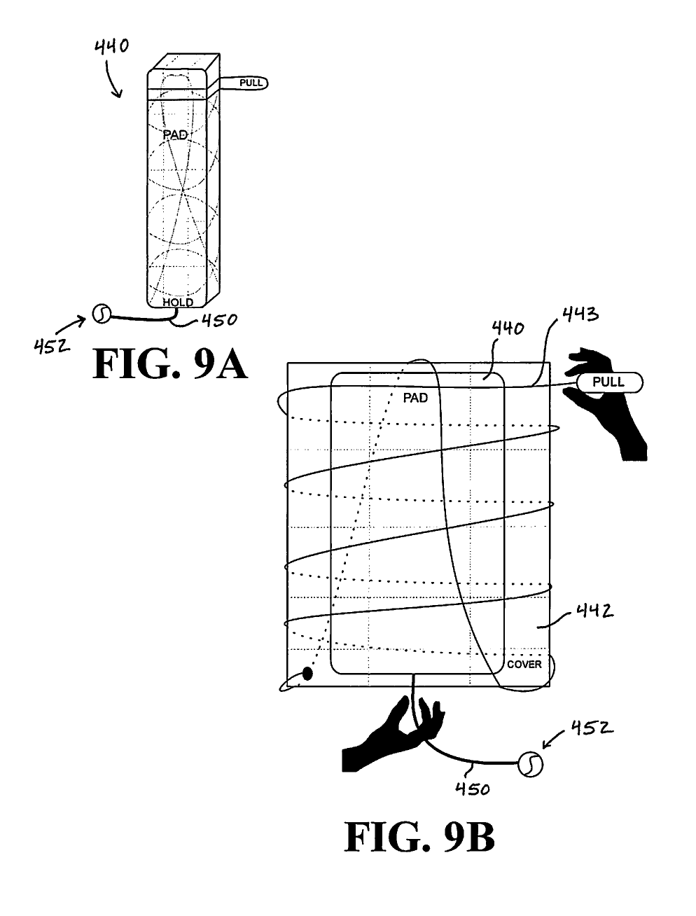

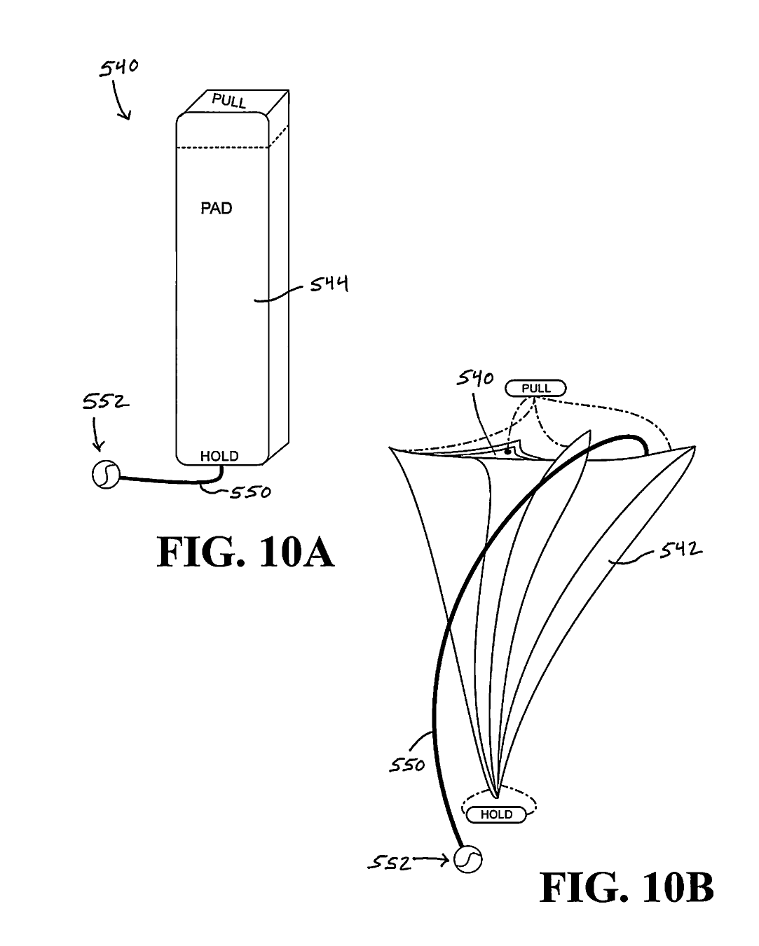

FIGS. 9A-B show greater details of the defibrillator pad 440. As depicted, the defibrillator pad 440 is preferably packaged or folded to provide for being contained within the case 412. Preferably, the pad 440 is capable of folding without compromising its intended functionality, for example to output electrical shock(s) and to receive EKG signals. In one example form, the pad 440 is releasably adhered to a cover member 442, wrapped with line or wrapping 443, and then folded to form the box-like package. Typically, wiring 450 extends from the pad and comprises a connector 452 for connecting to a connector (unshown) of the two respective pieces of the back half 416. As such, when it is determined that the pads 440 are to be replaced, the connector 452 provides for removing the expired or used pads from the back half 416 and removably attaching new pads 440. To use the pad 440, the bottom of the package is held with one hand (generally adjacent the wiring 450), and the wrapping 443 is pulled, which unwinds or removes the wrapping 443 therefrom. The cover 442 can then be removed to provide for removably attaching the pad 440 to the patient.

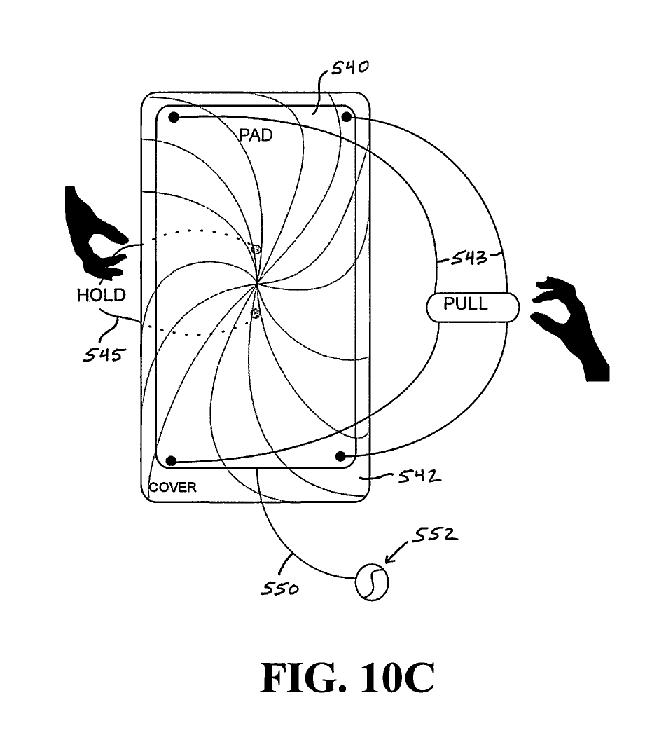

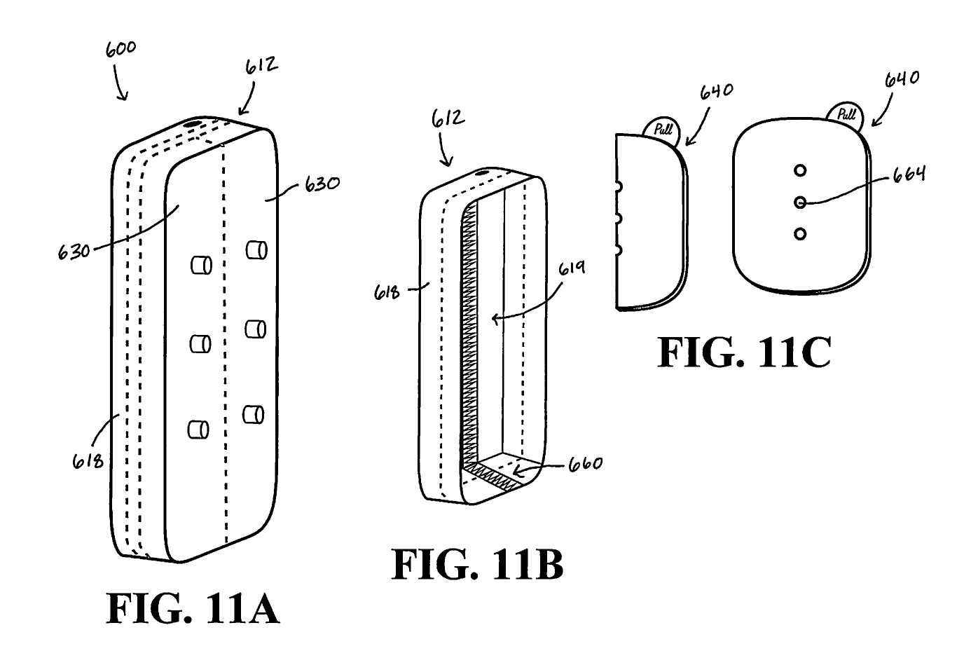

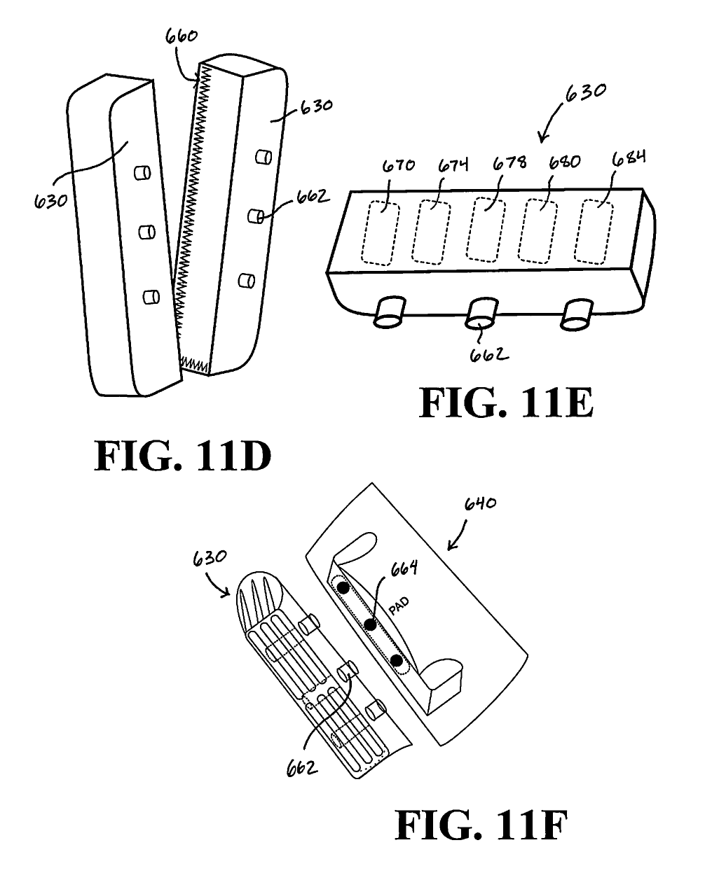

FIGS. 10A-C show another example embodiment of the defibrillator pads 540. As depicted, the defibrillator pad 440 (and cover 542 adhesively attached thereto) is generally folded similarly to a folded parachute. Typically, the folded pad 540 and cover 542 are packaged within a box-like member 544. As depicted in FIG. 10C, the two pull lines 543 are removably secured to the pad 540 and another pull line 545 is secured to the cover 542. In one example form, an operator pulls the two pull lines 543 while holding the pull line 545 to unfold the pad 540 and cover 542. When ready to use, the cover 542 can be removed from the pad 540.

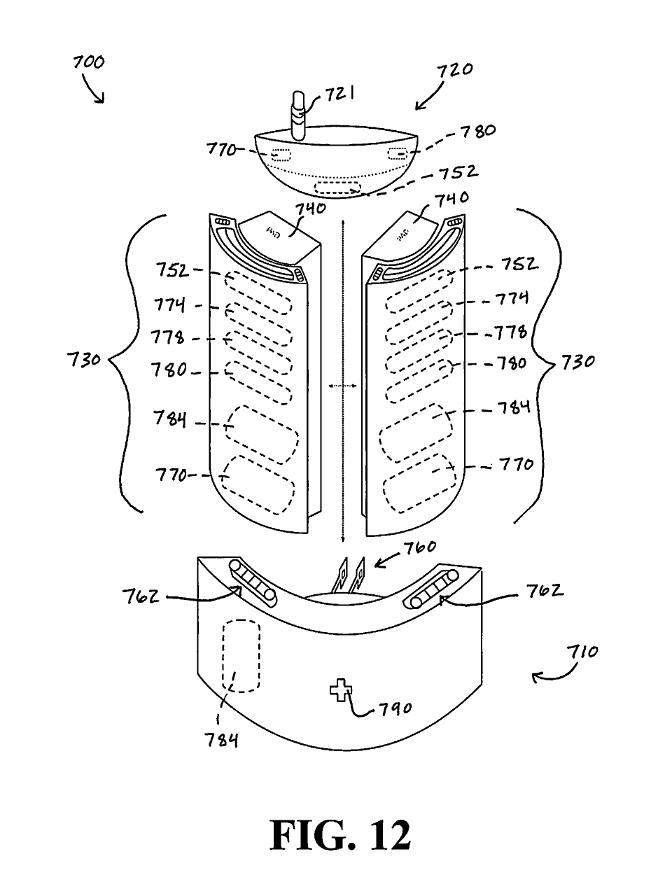

In additional example embodiments, the automated defibrillator attachment can connect to the electronic device D wirelessly. For example, the electronics module can comprise at least one of a plurality of forms of wireless communication means for wirelessly connecting to the defibrillator attachment wherein the EKG signals from the patient can be analyzed and processed by the supervisory software (sent wirelessly), and varying upon recognition of a shockable rhythm, output a signal (wirelessly) to the electronics module to provide at least one electrical shock. Example forms of wireless communication means may include USB, Bluetooth, WiFi, IR, or others as desired. Thus, when the defibrillator attachment is connected to the electronic device wirelessly, it is preferred that the energy storage element supplies the electrical power to provide for at least one electrical shock. For example, FIGS. 11A-F show a defibrillator attachment 600 combined with a releasable case 612. An outer shell 618 is provided for at least partially containing the electronic device D therein and defines a generally rearward-facing receptacle or reservoir 619 (see FIG. 11B) for containing the defibrillator pads 640 and two removably coupleable electronics modules 630. Preferably, the two electronics modules 630 each comprise an energy storage element 670, a shock delivery module 674, a signal processing electronics module 678, a control electronics module 680 a power management module 684, and at least one form of wireless communication means. In example forms, the pads 640 are folded in half when stored within the receptacle 619 and each comprise a plurality of female connectors 664 for cooperative engagement with a plurality of male connectors or electrode clip rivets 662 formed on the electronics modules 630. In one form, the male connectors 662 cooperatively engage the female connectors 664 of the pads 640, which allows the modules to electrically couple to the pads 640. In one example form, the top and bottom male connectors 662 are extensions of the shock delivery module 674 and the middle male connector 662 is an extension of the signal processing electronics module 678. Preferably, the number, shape, size, orientation, position, etc. of the male connectors 662 (and corresponding female connectors 664) can be chosen as desired. Further, it is preferred that the one or more male connectors 662 can be extensions of one or more components or modules as described herein.

Preferably, as similarly described above, the case 612 comprises a universal charging connector (having a male and female end, unshown) for connecting to the electronic device D when the device D is contained within the case 612, for example wherein the electronic device D while being at least partially housed within the case 312 is connected to the male end and wherein the female end can receive a connector (unshown) that is supplying electrical current. Additionally, a universal charging connector 660 (see zig-zag lines) is provided within the receptacle 619 for cooperative engagement with universal charging connectors 660 provided on each of the electronics modules 630 (see FIG. 110). Thus, when the electronics modules 630 are removably attached to the case 612 and contained within the receptacle 619, the connector (unshown) attached to the electronic device (e.g., for charging the device's D battery) can also supply power to the energy storage elements 670 of each of the electronics modules 630.

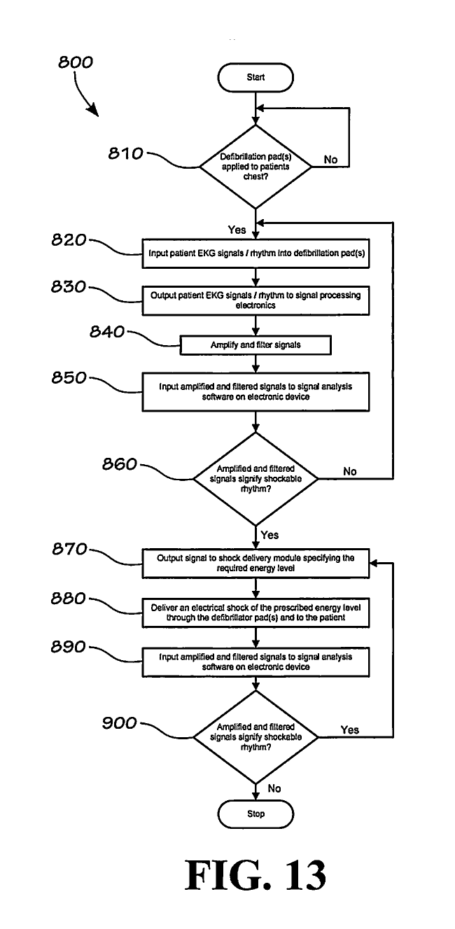

FIG. 12 shows a defibrillator attachment 700 according to another example embodiment of the present invention. As depicted, the defibrillator attachment is generally separable into four components including a base 710, a pair of electronics modules 730 (each comprising a defibrillator pad 740), and a connector 720 for connecting to an electronic device D. Preferably, as will be described below, each of the four components have the capability to wirelessly communicate therebetween. The base 710 generally includes a generally centrally-positioned universal charging connector 760 for connecting to a wall power outlet or other power outlet, a pair of male universal charging connectors 762 for cooperatively coupling to female universal charging connectors 764 (unshown) of the electronics modules 730, a power management module 784, and a charge level indicator 790. Preferably, the charge level indicator 790 can display a plurality of colors, on-off sequences, etc. to inform the operator as to the status of the defibrillator attachment 700. For example, a solid green light may indicate that the attachment 700 is fully charged and ready to use and a flashing red light may indicate that the device is charging or may need maintenance prior to being used. In one form, the power management module 784 controls and manages the power throughout the entire system.

The electronic modules 730 generally removably couple to the base 710 and each comprise an energy storage element 770, a shock delivery module 774, a signal processing electronics module 778, a control electronics module 780, a power management module 784 and wireless communication means 752. Preferably, each electronic module 730 comprises a defibrillator pad 740 affixed thereto. As described above, each electronics module 730 preferably comprises a cooperating female universal charging connector 664 (unshown) for coupling to the male charging connector 762, for example to allow for electrical current entering in the centrally-positioned charging connector 760 to supply electrical current to each of the energy storage elements 770 of each of the electronics modules 730. Generally, the power management modules 784 of each of the electronics modules 730 generally focuses on the power management for its respective module.

The connector 720 generally couples to the electronic modules 730 and generally comprises a connector 721, an energy storage element 770, a control electronics module 780, and wireless communication means 752. Preferably, the connector 720 is capable of connecting to the electronic device D via the connector 721 wherein the wireless communications means 752 communicates with the wireless communication means 752 of each of the electronics modules 730. Optionally, the connector 720 is omitted and the electronic device D can connect wirelessly to each of the electronics modules 730. In one example form, the control electronics module 780 of the connector 720 manages the entire power management system to ensure each of the components are working properly and are capable of holding an electrical charge. As similarly described above, the connector 720 is capable of communicating with the supervisory software of the electronic device D to provide for defibrillation. Optionally, the supervisory software is contained (e.g., zipped or downloaded thereto) within the connector 720 (or optionally the electronics modules 730 or base 710) and automatically downloaded to the electronic device D when the electronic device D is connected to the connector 720. Thus, in some example forms, some components of the defibrillator attachment can comprise computer readable media or memory for providing or delivering the supervisory software to the electronic device D.

Preferably, the cases as described herein can be constructed of one or more materials including plastic, metal, wood, composite, polymers, natural materials, synthetic materials, combinations thereof, and/or other materials as desired.

In additional example embodiments, the present invention relates to a defibrillation system having a first component and a second component. The first and second components are separate from each other. The first component comprises control software that is stored in memory, and the second component comprises electronics for permitting communication with the control software of the first component. The first component is generally in the form of an electronic device, and the second component is generally in the form of an automated external defibrillator attachment. The automated defibrillator attachment comprises an electronics module and defibrillator pads or electrodes. The first and second components can connect to each other by a wired connection or a wireless connection. Optionally, the second component comprises control software that is stored in memory within the electronics module. Thus, in some example forms, the first component receives the control software from the second component.

In additional example embodiments, the supervisory software can be utilized through one or more applications available on the electronic device, for example an internet browser (e.g., Safari, FireFox, etc.) or other application. In yet another example embodiment of the present invention, the delivery of therapy to the patient is generally independent from the electronic device D such that the electronic device D assists the user/operator in interfacing or acquiring knowledge about the signals being input and output from the defibrillator attachment. Thus, in some example forms, the defibrillator attachment or electronics module can operate independently of the electronic device D. In another example embodiment, the time-constrained components (e.g., to ensure the at least one electrical shock is delivered at the appropriate time) are configured to be housed within the defibrillator attachment or electronics module(s).

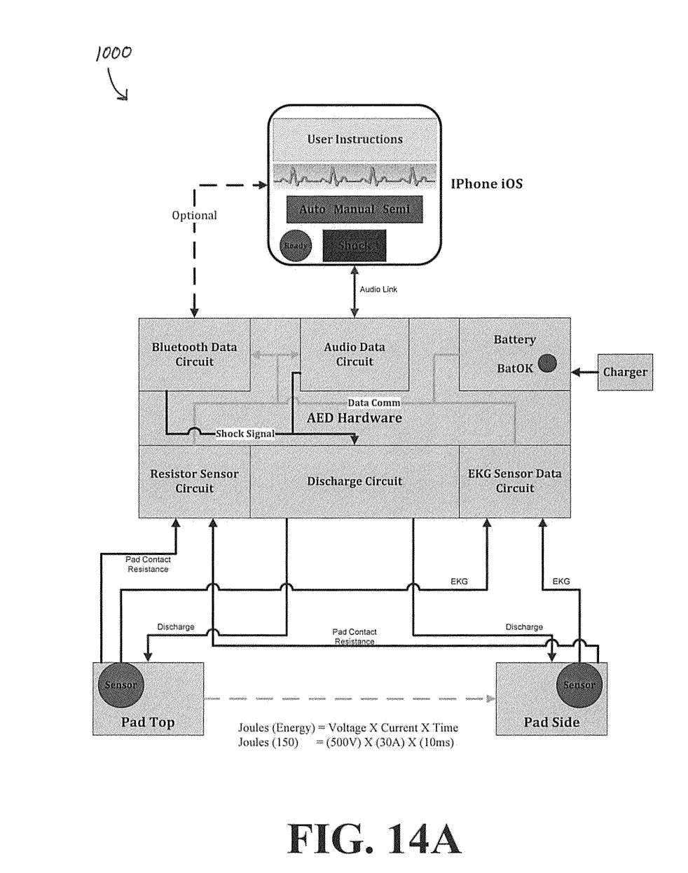

FIG. 13 shows a flow diagram detailing an operation process 800 of the automated defibrillator attachment according to another example embodiment of the present invention. After the connector is connected to the receptacle of the electronic device (or wirelessly) and the supervisory software is running on the electronic device, the process 800 can begin. At 810, the supervisory software determines if the electrodes or defibrillation pads are connected to the patient's chest. If the electrodes are connected to the patient's chest, the EKG signals or rhythm of the patient are input into the defibrillation pads (shown as 820). At 830, the EKG signals are output to the signal processing electronics of the electronics module. At 840, the signal processing electronics module amplifies and filters the EKG signals. At 850, the amplified and filtered signals are input into the signal analysis or supervisory software on the electronic device. The supervisory software then analyzes and processes the amplified and filtered signals and determines if they signify a shockable rhythm (shown as 860). If it is determined that the signals signify a shockable rhythm, then at 870, the supervisory software outputs a signal to the shock delivery module specifying the required energy level. Then, at 880, the shock delivery module in conjunction with the energy storage element produces an electrical shock of the prescribed energy level through the electrodes and to the patient. At 890, the amplified and filtered input signals (EKG signals) are again input into the signal analysis software on the electronic device, for example, to check for a successful electrical shock. The supervisory software then analyzes and processes the amplified and filtered signals and determines if they still signify a shockable rhythm (shown as 900). If it is determined that the signals signify a shock able rhythm, the process goes back to 870 wherein the supervisory software outputs a signal to the shock delivery module specifying the required energy level (and delivering the shock at 880). Otherwise, if the supervisory software determines that the amplified and filtered signals do not signify a shockable rhythm, process 800 ends. Optionally, the supervisory software can detect a normal rhythm of the patient. When detecting a normal rhythm of the patient, a queue can be given to the user or operator of the electronic device to check for a pulse of the patient.

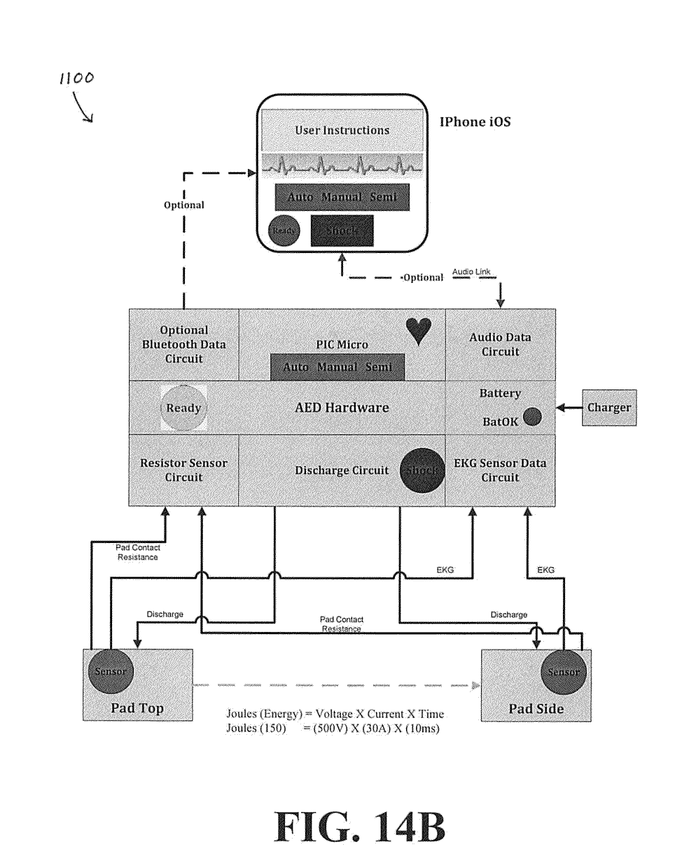

FIGS. 14A-B show schematic diagrams 1000 and 1100 of the defibrillator attachment according to additional example embodiments of the present invention.

In another example embodiment, the present invention relates to a method of treating a patient undergoing cardiac arrest. The method comprises providing an electronic device; providing a defibrillator attachment, the defibrillator attachment including an electronics module and at least two electrodes; connecting the defibrillator attachment to the electronic device; applying the at least two electrodes to the patient; receiving EKG signals from the patient; and outputting at least one electrical shock from the defibrillator attachment to the at least two electrodes.

While the invention has been described with reference to preferred and example embodiments, it will be understood by those skilled in the art that a variety of modifications, additions and deletions are within the scope of the invention, as defined by the following claims.

What is claimed is:

1. An automated external defibrillator attachment for an electronic device, the electronic device comprising an energy source and at least one receptacle, the attachment comprising:
an electronics module;
at least two electrodes extending from the electronics module for connecting to a patient; and
a connector extending from the electronics module for coupling to the at least one receptacle of the electronic device.

2. The automated defibrillator attachment of claim 1, wherein the at least two electrodes are configured for application to a patient in cardiac distress to provide at least one therapeutic electrical shock to the patient.

3. The automated defibrillator attachment of claim 2, wherein the at least two electrodes providing the at least one electrical shock is supplied from the energy source of the electronic device.

4. The automated defibrillator attachment of claim 3, wherein the electronics module comprises a current boosting element for receiving energy from the energy source of the electronic device and outputting the at least one electrical shock.

5. The automated defibrillator attachment of claim 4, wherein the energy level of the at least one electrical shock is between 120-200 joules.

6. The automated defibrillator attachment of claim 4, wherein the current boosting element is selected from a supercapacitor, a battery or a combination thereof.

7. The automated defibrillator attachment of claim 2, wherein the at least two electrodes can provide at least one electrical shock that is supplied from an external energy source.

8. The automated defibrillator attachment of claim 7, wherein the electronics module comprises an energy storage element for outputting the at least one electrical shock of an amplitude sufficient to treat a person in cardiac distress.

9. The automated defibrillator attachment of claim 8, wherein the energy delivered by the at least one electrical shock is between 120-200 joules.

10. The automated defibrillator attachment of claim 1, wherein the electronic device is a smart phone.

11. A portable external defibrillator attachment for an electronic device, the electronic device comprising at least one receptacle and a signal analysis software, the attachment comprising:
an electronics module comprising an energy storage element, a shock delivery module, signal processing electronics module, control electronics module, and power management electronics module;
at least two electrodes extending from the electronics module for connecting to a patient;
a connector extending from the electronics module for being received by the at least one receptacle to communicate with the electronic device; and
a recharging connector.

12. The portable external defibrillator attachment of claim 11, wherein the at least two electrodes can receive electrocardiographic signals from a patient.

13. The portable external defibrillator attachment of claim 12, wherein the electrocardiographic signals received by the at least two electrodes are input to the signal processing electronics of the electronics module to be amplified and filtered.

14. The portable electronic defibrillator attachment of claim 13, wherein the amplified and filtered signals are output from signal processing electronics and input to the electronic device to be processed by the signal analysis software to determine whether the patient is in a life threatening arrhythmia.

15. The portable external defibrillator attachment of claim 11, wherein the shock delivery module receives energy from the energy storage element to deliver at least one electrical shock through the at least two electrodes and to the patient.

16. The portable external defibrillator attachment of claim 11, wherein the recharging connector can be connected to a power outlet to charge the energy storage element.

17. A defibrillator attachment for connecting to an electronic device, the electronic device comprising a receptacle and software for both guiding a user, performing analysis of an input signal, and outputting an output signal, the attachment comprising: defibrillator pads, a signal conditioning subsystem, and a shock delivery subsystem, the defibrillator pads connected to the signal conditioning subsystem, the signal conditional subsystem connected to the electronic device, and the shock delivery subsystem connected to the electronic device and the defibrillator pads.

18. The defibrillator attachment of claim 17, wherein the signal conditioning subsystem receives electrocardiographic signals from a patient that comprise the input signals to be analyzed by the software to determine if the patient is in a life threatening arrhythmia.

19. The defibrillator attachment of claim 18, wherein the output signal is received by the shock delivery subsystem to trigger outputting at least one electrical shock to the defibrillator pads.

20. The defibrillator attachment of claim 19, wherein the shock delivery subsystem receives energy from an energy storage element to deliver at least one electrical shock through the defibrillator pads and to the patient.

21. A system comprising the defibrillator attachment of claim 17 in combination with an electronic device, the electronic device comprising a smart phone comprising a processor, computer readable memory, input means, output means, and control software loaded into said computer readable memory and executable by said processor to control the signal conditioning subsystem and the shock delivery subsystem.

22. A method of treatment utilizing the system of claim 21, said method comprising applying the defibrillator pads to a human or animal patient, and delivering at least one electrical shock to the patient, the at least one electrical shock being controlled by the electronic device in combination with the defibrillator attachment.

23. An automated external defibrillator comprising:
an electronic device comprising a processor and at least one coupling, and signal analysis software executable by the processor;
an electronics module comprising an energy storage element, a shock delivery module, a signal processing electronics module, a control electronics module, and a power management electronics module;
at least two electrodes extending from the electronics module for connecting to a patient; and
a connector extending from the electronics module for connection with the at least one coupling to communicate with the electronic device.

24. The automated external defibrillator of claim 23, wherein the electronic device is selected from a cell phone, a smart phone, an MP3 player, and electronic music player, a video player, an electronic reader, a tablet computer, or a handheld game device.

25. The automated external defibrillator of claim 24, wherein the electronic device is a smart phone.

26. A method of treating a patient undergoing cardiac arrest comprising:
providing an electronic device;
providing a defibrillator attachment, the defibrillator attachment comprising an electronics module and at least two electrodes;
connecting the defibrillator attachment to the electronic device;
applying the at least two electrodes to the patient;
receiving EKG signals from the patient; and
outputting at least one electrical shock from the defibrillator attachment to the at least two electrodes.

27. The method of claim 26, wherein the electronic device is a smart phone.
